(19) United States
(12) Patent Application Publication
(10) Pub. No.: US 2025/0152201 A1
(43) Pub. Date: May 15, 2025

(54) MULTI-AXIAL FOOT CORRECTION SYSTEM

(71) Applicant: DePuy Synthes Products, Inc., Raynham, MA (US)

(72) Inventors: Scott P. Lavoritano, West Chester, PA (US); Bernd Giso Gutmann, Hassloch (DE); Vaughn Charlton, III, Landenberg, PA (US)

(21) Appl. No.: 18/507,727

(22) Filed: Nov. 13, 2023

(51) Int. Cl. A61B 17/62; A61B 34/10

(52) U.S. Cl. CPC A61B 17/62; A61B 34/10; A61B 2034/105

(57) ABSTRACT

Described are methods and systems for correcting a foot of a patient in need thereof, comprising a bone fixation device comprising a first ring and a second ring connected by at least two interposed struts, each strut having a respective adjustable length, a display, and a processor configured to receive an indication of an orientation of at least one of the first and second rings with respect to a transverse plane of the patient's foot, receive an indication of which of the first ring or the second ring is a reference ring, receive initial lengths of each strut, receive an indication of an initial anatomical position of at least one bone in the patient's foot, receive an indication of a planned anatomical position of the bone in the patient's foot, and generate a strut adjustment plan relative to the reference ring for changing the length of at least one strut, thereby moving the bone toward the planned anatomical position.

### BACKGROUND

Ring fixation systems typically comprise a pair of rings spaced apart by a plurality of adjustable length struts. When separated bone fragments are physically attached to respective rings, such as by transfixion wires, Schanz pins, or connection elements, planned movements of the struts can be used to align the bone fragments. For example, even though an individual strut may only increase in length, decrease in length, or remain static, together, the struts may cooperate to produce a net rotation, translation, etc. with respect to a reference bone fragment.

Ring fixation frames are most commonly applied to the tibia, where axis determinations are relatively straight forward. As the complexity of fracture management and deformity correction grows, computer-assisted ring fixation systems, such as the MAXFRAME™ Multi-Axial Correction System, are increasingly important.

However, when treating a foot, it can be appreciated that movement between bone fragments should be accounted for in the transverse plane (with possible dorsal or plantar movement), the sagittal plane (with possible medial or lateral movement), and the frontal plane (with possible hindfoot or forefoot movement). As may be appreciated, a user (e.g., a surgeon) may find particular benefits from computer-assisted ring fixation systems which can be applied to the foot.

### SUMMARY

Described are methods and systems for correcting a foot of a patient in need thereof, comprising a bone fixation device comprising a first ring and a second ring connected by at least two interposed struts, each strut having a respective adjustable length, a display, and a processor configured to receive an indication of an orientation of at least one of the first and second rings with respect to a transverse plane of the patient's foot, receive an indication of which of the first ring or the second ring is a reference ring, receive initial lengths of each strut, receive an indication of an initial anatomical position of at least one bone in the patient's foot, receive an indication of a planned anatomical position of the bone in the patient's foot, and generate a strut adjustment plan relative to the reference ring for changing the length of at least one strut, thereby moving the bone toward the planned anatomical position.

### BRIEF DESCRIPTION OF THE DRAWINGS

FIG. 1A is a schematic of a graphical user interface (GUI) representation of a patient foot in a bone fixation device in a frame on axis orientation.

FIG. 1B is a schematic of a GUI representation of a patient foot in a bone fixation device in an ankle frame orientation.

FIG. 2 is a flowchart for determining a strut adjustment plan for a bone fixation device.

FIG. 3 is another flowchart for determining a strut adjustment plan.

FIG. 4 is another flowchart for determining a strut adjustment plan.

FIG. 5 is yet another flowchart for determining a strut adjustment plan.

### DETAILED DESCRIPTION

Described are methods and systems for correcting a foot of a patient in need thereof, comprising a bone fixation device comprising a first ring and a second ring connected by at least two interposed struts, each strut having a respective adjustable length. For example, the bone fixation device may be used to correct bone fractures, align (e.g., rotate and/or translate) bone segments (hereinafter referred to as bone fragments) resulting from osteotomy, or to correct soft tissue contractures.

The bone fixation device comprises a pair of rings (e.g., full rings, ⅝ rings, bridging plates, and foot plates), which may be the same or different, and a plurality of interposed struts (e.g., quick adjust and/or standard). In some embodiments, six struts are interposed between the first and second rings. In some embodiments, the bone fixation device comprises a third ring and a total of twelve struts, wherein six struts are interposed between the first and second rings, and wherein six struts are interposed between the second and third rings.

Other optional components include open wire posts and connection plates. These components may be used to create many frame configurations to address a wide variety of indications. Typically, such hardware is operably connected to a controller comprising a processor and memory storing software to aid a user (e.g., a surgeon) to create a patient treatment plan, which comprises a detailed plan for required strut adjustments to resolve the patient's unique circumstances.

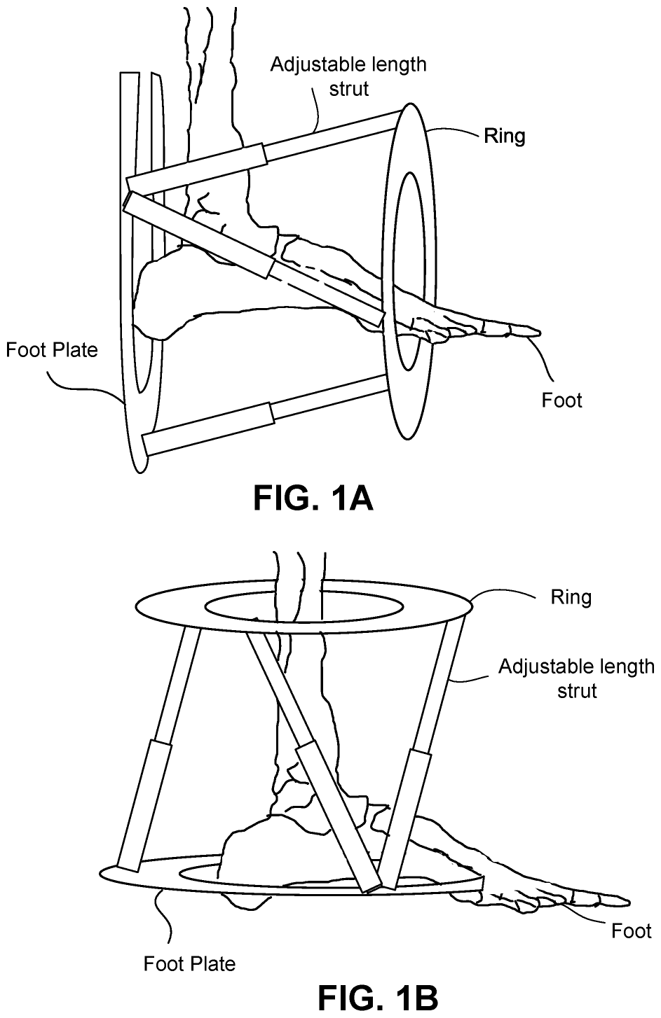

The controller may be operably connected to a display, for example, to aid input of parameters. For example, the controller may receive an indication of an orientation of at least one of the first and second rings with respect to a transverse plane of the patient's foot. The controller may generate a graphical user interface (GUI) representation of a patient foot in a bone fixation device for display on the display. For example, FIG. 1A is a schematic of a GUI representation in a frame on axis orientation (ring orientation is perpendicular to the transverse plane) and FIG. 1B is a schematic of a GUI representation of a patient foot in a bone fixation device in an ankle frame orientation (ring orientation is parallel to the transverse plane). It is understood that the GUI may be representation of a bone model, as will be described, such as a 3-dimensional (3D) bone model of the patient's specific anatomy. This model may be built using image data of the patient. Examples of image data include stored images. The image data may comprise computed tomography (CT), magnetic resonance imaging (MRI), or other three-dimensional images. The image data may comprise unprocessed data or may be processed or segmented to show boundaries between different tissues (bone, nerve, muscle, etc.). The image data may comprise two-dimensional (2D) images, such as X-rays, or 2D images merged to afford a 3D image (e.g., fluoroscopy), or video images (e.g., from a TELIGENT camera, endoscopes, microscopes, etc.). The image data may be provided to the controller in a predetermined format, such as Digital Imaging and Communications in Medicine (DICOM) format. In a preferred embodiment, the images are X-ray images. As may be appreciated, there are clinically relevant radiological views. For example, a user (such as a surgeon or other medical professional) may prefer an antero-posterior (AP) view, a postero-anterior (PA) view, or a lateral view. From that general view (or perspective), a user might have ordered acquisition of images along multiple minor axes. For example, in an AP view, a user might order an X-ray along a first axis, and also an X-ray along a second axis 10-15 degrees off of the first axis in relatively the same plan. In practice, this allows a different perspective and potentially the ability to view different anatomy, but still within the same view. Stated differently, a user might order an image of the patient's foot in a first axis (e.g., such as an AP of the foot) and an image of the patient's foot in a second axis (e.g., such as an AP of the deformity), wherein both images are in the same view, but have different axes.

As can be appreciated, the controller may receive any number of indications about the patient, the indication, the bone fixation device, etc. For example, the controller may receive an indication of which of the first ring or the second ring is a reference ring (e.g., hindfoot or forefoot). The controller may be configured to store the reference ring indication and use the reference ring indication to generate subsequent additional planning screens.

The controller may receive indications of types or initial lengths of each strut. The controller may receive indications of an identifier for each of the struts. The controller may be configured to receive an indication regarding a change in the length of at least one strut and use the information to update the strut adjustment plan. The controller may be configured to receive an indication regarding a strut swap.

The controller may receive an indication of an initial anatomical position of at least one bone in the patient's foot. The controller may receive an assignment of a plurality of bones of the foot to the reference ring. Preferably, there is no limit on which bones can be assigned to which ring. Assignment is typically based on the implant selection, and which ring the implant is attached to mechanically, thereby determining the anatomic location of distraction and deformity correction. In fact, it is advantageous (although more complex for the controller) to assign a plurality of bones to the reference ring as it may increase a surgeon's awareness to movement of bones that are not directly mechanically fixed to the reference ring but will move based on their attachment to other bones. This may improve patient outcomes. The controller may receive an indication of a planned anatomical position of the bone in the patient's foot.

In some embodiments, the bones may be individually assigned to the forefoot and hindfoot fragment, thereby creating a soft correction without an osteotomy step.

The controller may be configured to receive an indication of at least one of a first center line and a second center line, a first reference point and a second reference point, or an indication of pronation or supination.

The controller may use the indications and generate a strut adjustment plan relative to the reference ring for changing the length of at least one strut, thereby moving the bone toward the planned anatomical position. The controller may display cut lines to represent different osteotomy locations. The controller may determine the bone based on an osteotomy selection. The controller may create a bone model. The bone model may adjust for articulation between the joints of the mid foot. The controller may use the planned anatomical position to perform a soft tissue correction (e.g., the methods and systems, in some embodiments, do not have an osteotomy step).

The controller may be configured to overlay at least one of a label or a measurement over an image of the patient's foot and the bone fixation device and display both on the display. The controller may be configured to overlay a normal anatomy overlay over the image, e.g., to help plan deformity correction.

The controller may be configured to divide a set of movements required to achieve the planned anatomical position into incremental subsets of movements over a period of time, compare each change in the length of at least one strut required in a subset to a predetermined value, and if the change in the length of the at least one strut is less than or equal to the predetermined value, store the strut adjustment plan.

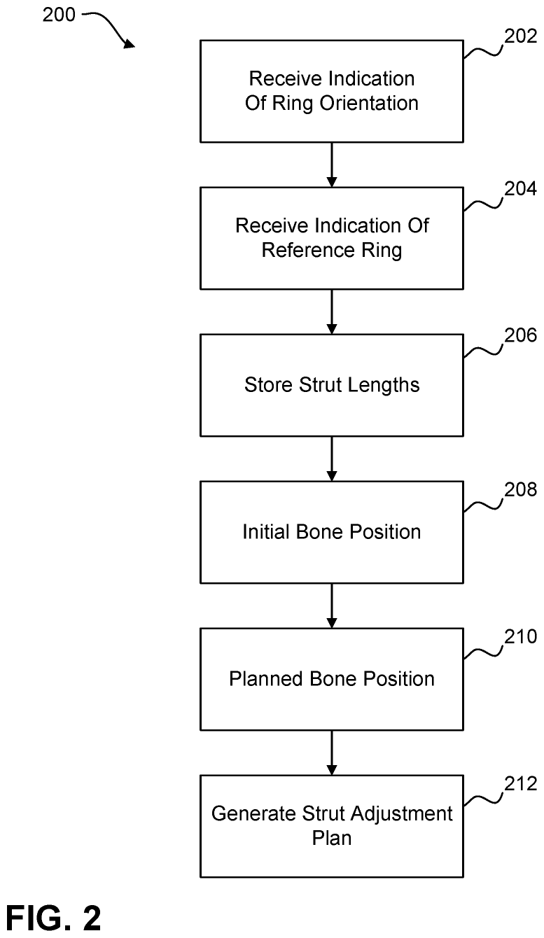

FIG. 2 is a flowchart for a method 200 performed by a controller, such as described above, for determining a strut adjustment plan for the bone fixation device. At step 202, the controller receives an indication of an orientation of at least one of the first and second rings with respect to a transverse plane of the patient's foot. At step 204, the controller receives an indication of which of the first ring or the second ring is a reference ring. At step 206, the controller receives an indication of initial lengths of each strut and stores the same. At step 208, the controller receives an indication of an initial anatomical position of at least one bone in the patient's foot. At step 210, the controller receives an indication of a planned anatomical position of the bone in the patient's foot (e.g., after osteotomy or a soft tissue correction). At step 212, the controller generates a strut adjustment plan based on indications from the preceding steps.

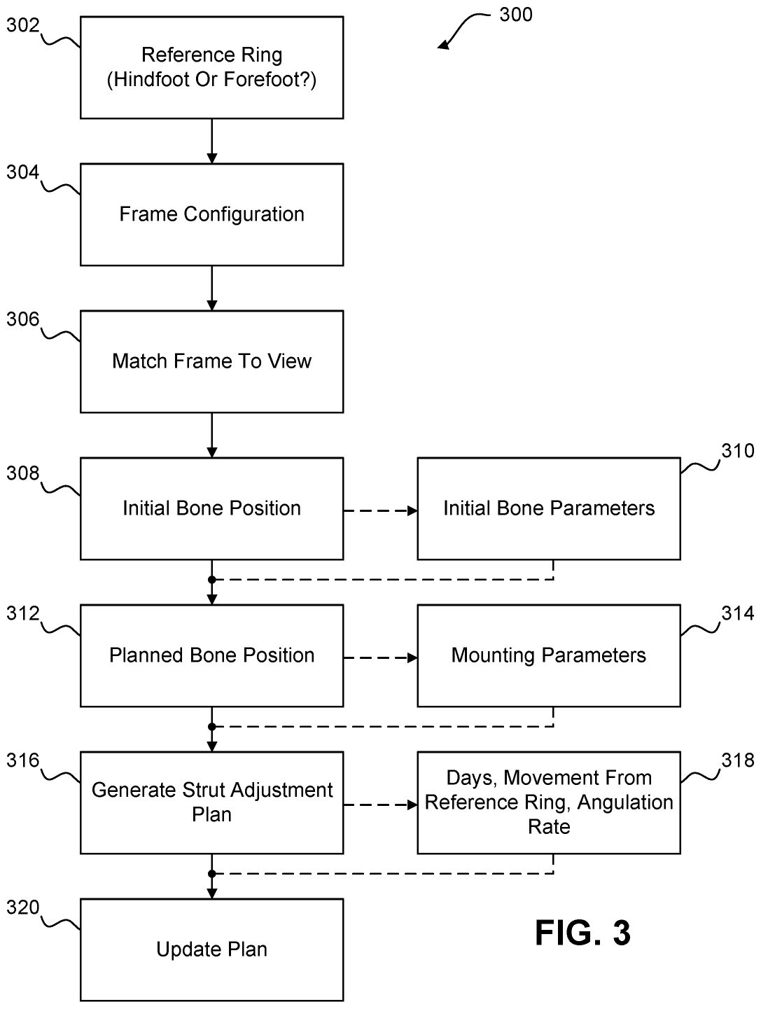

FIG. 3 is a flowchart for a method 300 performed by a controller, such as described above, for determining a strut adjustment plan for the bone fixation device. At step 302, the controller receives an indication of which of the first ring or the second ring is a reference ring. At step 304, the controller receives an indication of the frame configuration (which may or may not include an indication of an orientation of at least one of the first and second rings with respect to a transverse plane of the patient's foot) and stores the same. At step 306, the controller matches the frame configuration to a view. At step 308, the controller determines an initial anatomical position of at least one bone in the patient's foot. At step 310, the controller determines and/or receives indications of initial bone parameters. At step 312, the controller receives an indication of a planned anatomical position of the bone in the patient's foot (e.g., after osteotomy or a soft tissue correction). At step 314, the controller determines and/or receives indications of initial bone parameters. At step 316, the controller generates a strut adjustment plan based on indications from the preceding steps. At step 318, the controller determines parameter(s) such as days of treatment, movement from the reference ring, and angulation rate. At step 320, the controller generates an updated strut adjustment plan based on the preceding steps.

The controller may be configured to receive an image of the patient's foot in a first axis (e.g., such as an AP of the foot), receive an image of the patient's foot in a second axis (e.g., such as an AP of the deformity), wherein both images are in the same view, determine a rotation to move from the initial anatomical position to the planned anatomical position, and determine changes in the struts' lengths to produce the correction (e.g., a rotation, a translation, etc.). In some embodiments, the controller may be configured to utilize additional views to create a 3-dimensional (3D) correction tool. For example, one view may be orthogonal, but the other view may be the best visualization of the deformity. The controller may receive a tracing of the bone in the view with the visualization, and the controller may determine how the tracing relates to the bone in the orthogonal view (e.g., to accurately understand and thus predict how to correct the deformity). In some embodiments, the controller may receive an image of the patient's foot in a third axis, wherein all three images are in the same view.

In a preferred embodiment, a pair of spaced apart fiducials (e.g., markers) may be affixed to the foot (e.g., a plantar surface of the foot) and the images taken. It is understood that, depending on the type of imaging, different fiducials. Preferably, the fiducials provide high contrast in the image. The controller may comprise a processor configured to determine a position of each fiducial and determine a distance between them. For example, the controller may determine a distance between the fiducials in the first axis image. The controller may determine a distance between the fiducials in the second axis image. The controller may compare the distance between the fiducials in the first axis image and distance between the fiducials in the second axis image. If the fiducials are not the same distance apart in the first axis image and the second axis image, the controller may use the images to better model the deformity. For example, fiducials may be used to determine center of rotation for the correction or calculate translations. The controller may determine a correction to move from the initial anatomical position to the planned anatomical position. The controller may determine changes in the struts' lengths to produce the correction.

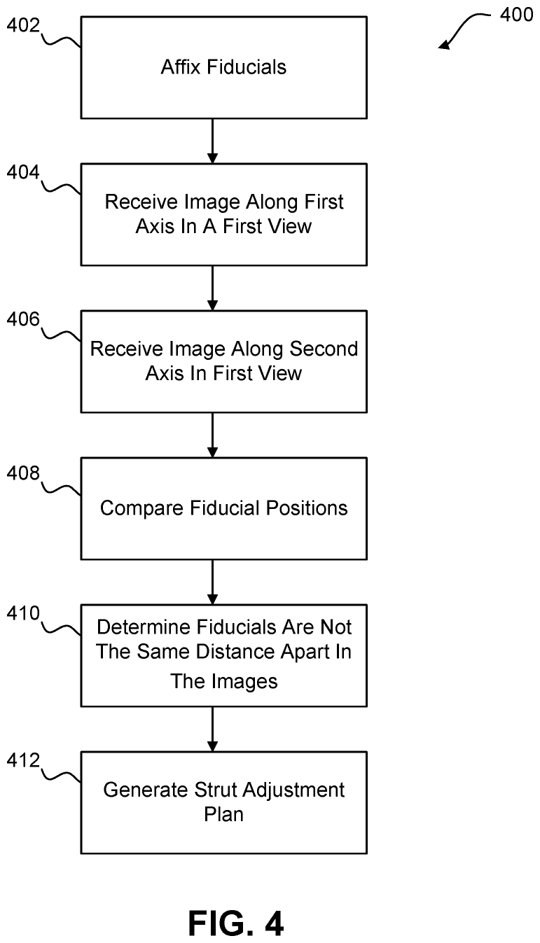

FIG. 4 is a flowchart for a method 400. At step 402, a pair of spaced apart fiducials may be affixed to the foot (e.g., a plantar surface of the foot). Steps 404-412 may be performed by a controller, such as described above, for determining a strut adjustment plan for the bone fixation device. At step 404, the controller receives an image of the patient's foot in a first axis (e.g., such as an AP of the foot). At step 406, the controller receives an image of the patient's foot in a second axis (e.g., such as an AP of the deformity). At step 408, the controller compares the images to determine if the fiducials are the same distance apart in the first axis image and the second axis image. At step 410, if the controller determines the fiducials are not the same distance apart in the first axis image and the second axis image, the controller generates a 3D model of the initial anatomical position of at least one bone in the patient's foot. At step 412, the controller generates a strut adjustment plan based on indications from the preceding steps.

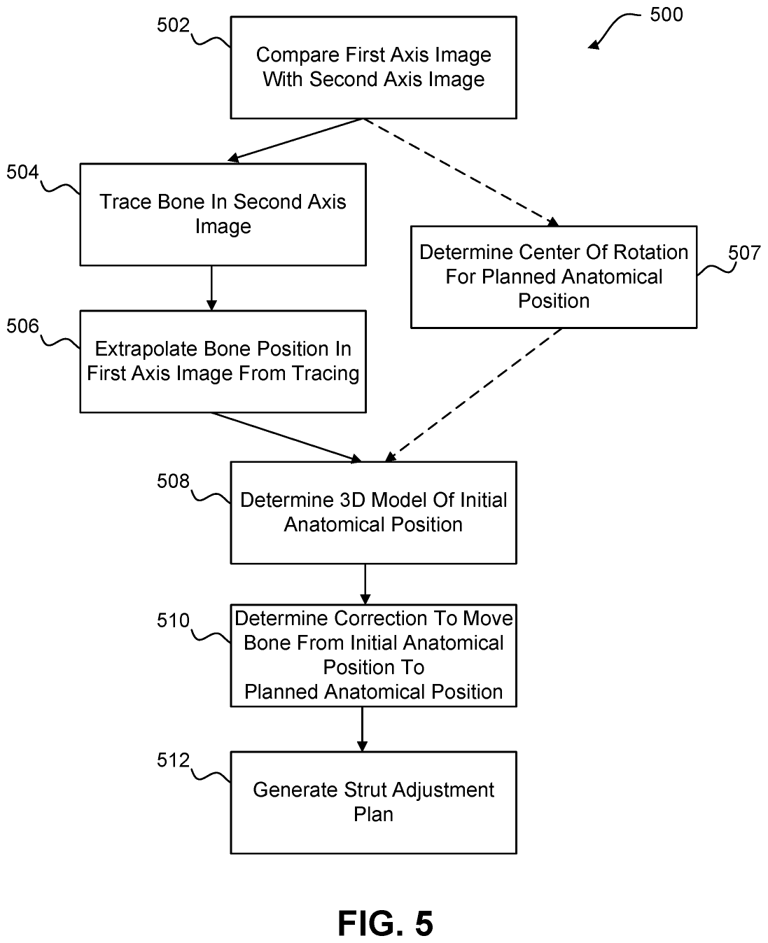

FIG. 5 is a flowchart for a method 500 that may be performed by a controller, such as described above, for determining a strut adjustment plan for the bone fixation device. For example, controller may be configured to receive an image of the patient's foot in a first axis (e.g., such as an AP of the foot), receive an image of the patient's foot in a second axis (e.g., such as an AP of the deformity), wherein both images are in the same view. At step 504, the controller receives a tracing of the bone in the second axis image (e.g., the image with view with the better visualization of the deformity). At step 506, the controller determines how the tracing relates to the bone in the first axis image. Alternatively, at step 507, the controller determines a center of rotation or a translation for the planned anatomical position from the comparison. At step 508, the controller generates a 3D model of the initial anatomical position of the at least one bone in the patient's foot. At step 510, the controller determines a rotation (or a translation) to move the bone from the initial anatomical position to the planned anatomical position. At step 512, the controller generates a strut adjustment plan based on indications from the preceding steps.

1. A system for correcting a foot of a patient in need thereof, comprising:
a bone fixation device comprising a first ring and a second ring connected by at least two interposed struts, each strut having a respective adjustable length;
a display; and
a processor configured to:
receive an indication of an orientation of at least one of the first and second rings with respect to a transverse plane of the patient's foot;
receive an indication of which of the first ring or the second ring is a reference ring;
receive initial lengths of each strut;
receive an indication of an initial anatomical position of at least one bone in the patient's foot;
receive an indication of a planned anatomical position of the bone in the patient's foot; and
generate a strut adjustment plan relative to the reference ring for changing the length of at least one strut, thereby moving the bone toward the planned anatomical position.

2. The system of claim 1, wherein the processor is further configured to store the reference ring indication and use the reference ring indication to generate subsequent additional planning screens.

3. The system of claim 1, wherein the processor is further configured to display cut lines to represent different osteotomy locations.

4. The system of claim 3, wherein the processor is further configured to suggest at least one cut line or joint line location.

5. The system of claim 1, wherein the processor is further configured to determine a first fragment and a second fragment of the bone based on an osteotomy selection.

6. The system of claim 1, wherein the processor is further configured to receive an assignment of a plurality of bones of the foot to the reference ring.

7. The system of claim 1, wherein the processor is further configured to overlay at least one of a label or a measurement over an image of the patient's foot and the bone fixation device.

8. The system of claim 1, wherein the processor is further configured to store at least one of a first center line and a second center line, a first reference point and a second reference point, or an indication of pronation or supination.

9. The system of claim 1, wherein the processor is further configured to store information regarding a change in the length of the at least one strut and use the information to update the strut adjustment plan.

10. The system of claim 1, wherein the processor is further configured to:
divide a set of movements required to achieve the planned anatomical position into incremental subsets of movements over a period of time;
compare each change in the length of at least one strut required in a subset to a predetermined value; and
if the change in the length of the at least one strut is less than or equal to the predetermined value, store the strut adjustment plan.

11. The system of claim 1, wherein the processor is further configured to:
receive an image of the patient's foot in a first axis;
receive an image of the patient's foot in a second axis, wherein both images are in the same view;
compare the images to determine a three-dimensional (3D) model of the initial anatomical position;
determine a rotation to move from the initial anatomical position to the planned anatomical position; and
determine changes in the struts' lengths to produce the correction.

12. The system of claim 11, wherein the processor is further configured to:
receive input comprising a tracing of the bone in the second axis image; and
extrapolate a position of the bone in the first axis image from the tracing.

13. The system of claim 11, wherein the processor is further configured to:
receive an image of the patient's foot in a third axis, wherein all three images are in the same view.

14. The system of claim 11, further comprising a pair of fiducials affixed to the foot, wherein the fiducials are spaced apart, and in the view, the fiducials are not the same distance apart in the first axis image and the second axis image.

15. The system of claim 14, wherein respective positions of the fiducials are used to determine a center of rotation or a translation for the planned anatomical position and/or to create a three-dimensional (3D) bone model.

16. The system of claim 1, wherein the bone fixation device comprises a third ring and a total of twelve struts, wherein six struts are interposed between the first and second rings, and wherein six struts are interposed between the second and third rings.

17. A system for correcting a foot of a patient, comprising:
a bone fixation device comprising a first ring and a second ring connected by at least two interposed struts, each strut having a respective adjustable length;
a display; and
a processor configured to:
receive an indication of which of the first ring or the second ring is a reference ring;
display a representation of the patient's foot in a first view;
display a representation of the patient's foot in a second view;
receive an indication of a three-dimension planar cut of at least one bone in the patient's foot to afford a first fragment and a second fragment; and
generate a strut adjustment plan relative to the reference ring for changing the length of at least one strut, thereby moving at least one of the fragments from an initial anatomical position toward a planned anatomical position.

18. The system of claim 17, wherein the processor is further configured to:
display cut lines to represent different osteotomy locations; or
suggest at least one cut line or joint line location.

19. The system of claim 17, wherein the processor is further configured to receive an assignment of a plurality of bones of the foot to the reference ring.

20. The system of claim 17, wherein the processor is further configured to:
receive an image of the patient's foot in a first axis with a pair of spaced apart fiducials affixed to the foot;
receive an image of the patient's foot in a second axis with the pair of fiducials affixed to the foot, wherein both images are in the same view and the fiducials are not the same distance apart in the apart in the first axis image and the second axis image;
determine a rotation to move from the initial anatomical position to the planned anatomical position; and
determine changes in the struts' lengths to produce the correction.
